(19) United States
(12) Patent Application Publication
(10) Pub. No.: US 2012/0289746 A1
(43) Pub. Date: Nov. 15, 2012

(54) PROCESS FOR WORKING UP AN ISOCYANATE-COMPRISING STREAM

(75) Inventors: Ulrich Penzel, Tettau (DE); Siegfried Richter, Schraden (DE); Ulrich Juedes, Schwarzheide (DE); Matthias Neumann, Maerkische Heide (DE); Hermann Lachmann, Schwarzheide (DE)

(73) Assignee: BASF SE, Ludwigshafen (DE)

(21) Appl. No.: 13/467,371

(22) Filed: May 9, 2012

Related U.S. Application Data
(60) Provisional application No. 61/483,774, filed on May 9, 2011.

(51) Int. Cl. C07C 209/62

(52) U.S. Cl. 564/393; 564/468; 564/455

(57) ABSTRACT

The invention relates to a process for working up a stream (3) comprising from 5 to 90% by weight of isocyanate by hydrolysis of the isocyanate by means of water in a reactor (1), in which the isocyanate-comprising stream (3) is introduced to the reactor (1) via a first inlet and the water is introduced via a second inlet, the isocyanate is reacted with the water in the reactor to form the corresponding: diamine, a diamine-comprising product mixture is taken off from the reactor (1) via a product outlet, wherein part of the product mixture is recirculated to the reactor and the isocyanate-comprising stream (3) is introduced into the recirculated product stream (7), with the isocyanate-comprising stream (3) being introduced continuously without interruption and the molar ratio of H-acid compounds of the hydrolyzate to isocyanate groups of the stream introduced being kept in the range from 10 to 200.

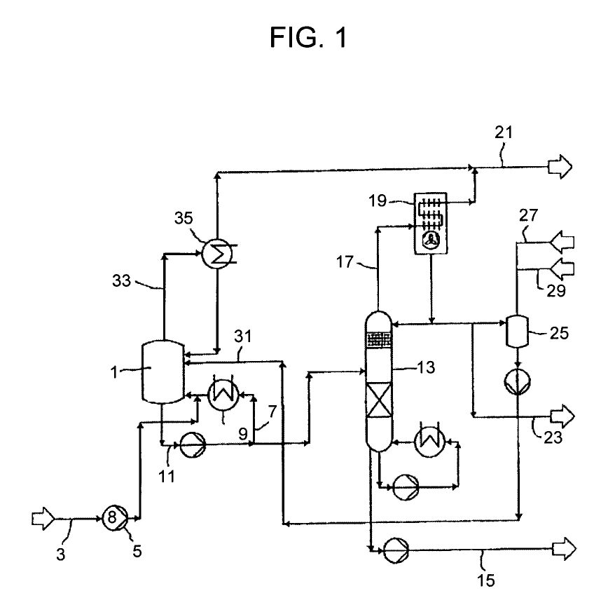

The invention relates to a process for working up an isocyanate-comprising stream by hydrolysis of the isocyanate by means of water in a fully backmixed reactor, in which
the isocyanate-comprising stream is introduced to the reactor via a first inlet and the water is introduced via a second inlet,
the isocyanate is reacted with the water in the reactor to form the corresponding diamine,
a diamine-comprising product mixture is taken off from the reactor via a product outlet.

Isocyanate is used in large amounts for producing polyurethanes. Isocyanate is usually prepared by reacting the corresponding diamine with phosgene. This process has been known for a long time. The diamine is usually reacted with phosgene in a conventional two-stage phosgenation. However, there are also other syntheses in which isocyanate is prepared by dissociation of a urethane, with the urethane being synthesized from diamine, urea and alcohol or in another way.

In all of these cases, there is a distillation step in which the isocyanate is separated from by-products at the end of the synthesis. The proportion of isocyanate in the high-boiling residue from this distillation step can be in the range from 5% by weight to 90% by weight. There is therefore a considerable economic incentive to utilize this residue as material.

DE-A 198 27 086 discloses a process in which the isocyanate from the distillation residue is reacted with water in a continuous or semicontinuous process in a backmixed reactor in the presence of hydrolyzate. Here, the distillation residue is reacted to give the corresponding diamine and carbon dioxide.

A process for preparing isocyanate is described in DE-A 10 2006 060 181. Here, the isocyanate is obtained by reacting diamine with phosgene. The isocyanate is purified by distillation and the distillation residue obtained in the distillation is hydrolyzed at a temperature of less than 230° C. and a pressure of less than 30 bar. The diamine obtained is subsequently recirculated to the reaction of diamine and phosgene to form isocyanate. To aid the reaction, bases, for example sodium hydroxide or potassium hydroxide solution, meta-TDA, ortho-TDA, ammonia or secondary compounds such as toluidines, aminomethylcyclohexanes or diaminomethylcyclohexanes, are added. The diamine is separated off from the resulting hydrolysis mixture by distillation.

The work-up by hydrolysis of a distillation residue obtained in the preparation of isocyanate is also described in DE-A 27 03 313. The hydrolysis of an isocyanate-comprising distillation residue by means of water to give diamine is also described in WO-A 00/68180. Here, a strong acid is added to keep the pressure in the reactor at a prescribed level.

A process for preparing monoamines, diamines and/or polyamines from carbodiimide groups and optionally other groups of isocyanate chemistry by hydrolysis with water is also described in DE-A 10 2004 011 320. Finally, the work-up of an isocyanate-comprising distillation residue by hydrolysis by means of water is also known from WO-A 2004/108656 and U.S. Pat. No. 4,137,266. However, according to U.S. Pat. No. 4,137,266, the solvolysis is not carried out by means of water but in the presence of aqueous ammonia.

A disadvantage of the known processes is that although the possibility of hydrolysis of an isocyanate-comprising distillation residue is mentioned in the prior art, there is no information as to how the isocyanate-comprising distillation residue can be fed without interruption to a continuously operating hydrolysis; the isocyanate-comprising distillation residue has to be introduced into a reaction mixture which comprises a mixture of amines and water and reacts vigorously with isocyanate, has a high temperature and is under a high pressure.

It is an object of the present invention to provide a process for working up an isocyanate-comprising stream by hydrolysis, which does not have the disadvantages known from the prior art.

The object is achieved by a process for working up a stream comprising from 5 to 90% by weight of isocyanate by hydrolysis of the isocyanate by means of water in a reactor, in which
the isocyanate-comprising stream is introduced to the reactor via a first inlet and the water is introduced via a second inlet,
the isocyanate is reacted with the water in the reactor to form the corresponding diamine,
a diamine-comprising product mixture is taken off continuously from the reactor via a product outlet,
   wherein part of the product mixture is recirculated to the reactor and the isocyanate-comprising stream is introduced into the recirculated product stream, with the isocyanate-comprising stream being introduced continuously without interruption and the molar ratio of H-acid compounds of the hydrolyzate to isocyanate groups of the stream introduced being kept in the range from 10 to 200.

For the purposes of the present invention, isocyanate is, for example, tolylene diisocyanate (TDI), hexamethylene diisocyanate (HDI), isophorone diisocyanate (IPDI), xylylene diisocyanate (XDI), dimethylpropane diisocyanate (DMPDI), 2-methylpentane diisocyanate (2-Me-PDI) or another isocyanate prepared by phosgenation of the corresponding amine or by a phosgene-free route. The corresponding diamine is in each case toluenediamine (TDA), hexamethylenediamine (HIDA), xylylenediamine (XDA), dimethyipropanediamine (DMPDA), 2-methylpentanediamine (2-Me-PDA) or another amine used for the phosgenation or phosgene-free isocyanate route. The process of the invention is particularly suitable for working up a TDI-comprising distillation residue.

By means of the process of the invention, the isocyanate-comprising distillation residue is introduced without interruption into the hydrolysis reactor. Introduction into the recirculated product stream additionally ensures that the inflow rate of the isocyanate-comprising distillation residue into the hydrolysis reactor is greater than the speed of the reaction front at the point at which the isocyanate is introduced into the aqueous hydrolyzate in the hydrolysis reactor. This prevents the ureas initially formed at the reaction front from blocking the inlet. In the course of the further reaction, the solid ureas initially formed are hydrolyzed further to aromatic amines with formation of carbon dioxide.

According to the invention, the molar ratio of H-acid compounds of the hydrolyzate to isocyanate groups of the stream introduced is kept in the range from 10 to 200. Particular preference is given to keeping the molar ratio of H-acid compounds of the hydrolyzate to isocyanate groups of the stream introduced in the range from 130 to 170. This has the advantage that many particularly small urea particles having a high total surface area are initially formed and these then react with dissolution in a solid-liquid reaction to form the corresponding amine and carbon dioxide. The oligomeric by-products of the isocyanate synthesis, e.g. biurets, allophanates, uretdiones, carbodiimides, uretonimines, isocyanurates, likewise comprised in the solids are likewise at least partly hydrolyzed to the corresponding amines. The molar ratio of the H-acid compounds in the aqueous hydrolyzate to isocyanate groups of the stream introduced is set by selection of the amounts of the isocyanate-comprising stream fed into the hydrolysis reactor and the amount of aqueous hydrolyzate recirculated to the reactor.

For the purposes of the present invention, H-acid compounds are, for example, water and/or amines.

The isocyanate-comprising distillation residue generally originates from the preparation of isocyanate used for producing polyurethanes. Isocyanate is usually prepared by reacting the corresponding diamine with phosgene. For this purpose, the diamine is usually reacted with phosgene in a two-stage phosgenation. The reaction of the diamine with phosgene is followed by a distillation step in which the isocyanate is separated off from by-products. The distillation residue formed here generally comprises from 5 to 90% by weight, preferably from 10 to 50% by weight, of isocyanate, from 10 to 95% by weight, preferably from 50 to 90% by weight, of ureas, tar-like oligomers of the isocyanate, urethanes, isocyanurates, biurets, allophanates, uretdiones, carbodiimide, uretonimines and other by-products.

The residue used for the hydrolysis can be taken from the bottom of the last work-up column or downstream evaporation apparatuses. The evaporation apparatuses can be, for example, falling film evaporators, a thin film evaporator, a forced circulation flash evaporator, a heated tube with a flash pot, a stirred vessel, a heated ball mill, a single-screw or twinscrew extruder, a fluidized bed, a paddle dryer, a 2-phase helical tube or any other desired apparatus. Neither the isocyanate content nor the consistency of the isocyanate residue, i.e. whether the residue is liquid or solid, and thus also the type of evaporation apparatus restrict the applicability of the process of the invention for working up the isocyanate-comprising stream. However, to improve the handleability of the isocyanate-comprising stream, it is advantageous if this is pumpable or suspendable.

To improve the handleability of the isocyanate-comprising stream further, it can also be mixed with a suitable organic solvent which does not react with the substances comprised in the stream, for example toluene, N-methylpyrrolidone, dimethylformamide, monochlorobenzene, dichlorobenzene and others.

The hydrolysis can be aided by the concomitant use of bases, for example alkali metal and/or alkaline earth metal hydroxides, for example sodium hydroxide, potassium hydroxide, magnesium hydroxide, calcium hydroxide, magnesium oxide, calcium oxide, ammonia or amines, and also acids such as hydrochloric acid, hydrobromic acid or sulfuric acid.

When alkali metal hydroxides are used, these are preferably used in an amount of from 0.5 to 5.0% by weight, based on the reaction mixture.

The use of catalysts as are customarily used for the dissociation of urethanes, for example salts of iron, zinc, tin and zirconium, likewise promote the hydrolysis.

The hydrolyzate is taken off continuously from the hydrolysis reactor and worked up. In the case of the preferred solvent-free process, the hydrolyzate consists of a single phase. The water used in excess is separated off first by distillation and the toluenediamine is subsequently separated off by distillation. When water-insoluble solvents are used, a major part of the water can be separated off by means of a preceding phase separation. The water which has been separated off can be returned to the hydrolysis.

The toluenediamine recovered by means of the hydrolysis is, after appropriate purification and work-up, introduced into the phosgenation reactor of the process for preparing tolylene diisocyanate or added to the reaction mixture leaving the hydrogenation reactor in the preparation of toluenediamine by hydrogenation of dinitrotoluene before the work-up to give pure toluenediamine. The latter process variant has the advantage that the separate work-up step for the hydrolyzate after the hydrolysis may be able to be dispensed with entirely. The by-products of the hydrolysis can then be discharged from the process together with the toluenediamine tar in the toluenediamine work-up.

To prevent solid urea formed in the region of the reaction front from blocking the feed lines, the flow rate of the stream introduced is preferably greater than the speed of the reaction front at the point of introduction. The reaction front moves in the direction of the stream introduced and is thus in a direction opposite to the flow direction of the stream.

The stream comprising tolylene diisocyanate is preferably introduced by means of a multihead positive displacement pump which is equipped with solids-tolerant inlet and outlet valves on the product side. The use of a multihead positive displacement pump having solids-tolerant inlet and outlet valves has the advantage that the stream being pumped has little pulsation and is physically steady.

The stream comprising tolylene diisocyanate is particularly advantageously introduced into the hydrolyzate via a mixing element, for example configured in the form of a Venturi nozzle, preferably using the hydrolyzate in the hydrolysis reactor as driving jet for the Venturi nozzle. The qualitatively good mixing of the isocyanate-comprising stream into an excess of H-acid compounds, for example amines and water, in a Venturi nozzle leads to the formation of many, particularly small solid particles, preferably ureas, having a large surface area. The formation of particularly small solid particles to be hydrolyzed leads to high conversions in the hydrolysis reaction at moderate residence times and temperatures.

To avoid formation of solid ureas and blockage of the reactor or lines during start-up and shutdown, it has been found to be advantageous to introduce an inert barrier liquid between the reactants during start-up and shutdown of the metering of the stream comprising tolylene diisocyanate into the reactor. Furthermore, it has been found to be advantageous to keep the pressure on the feed side of the tolylene diisocyanate above the pressure on the hydrolyzate side at the point of introduction.

The hydrolysis of tolylene diisocyanate by means of water to form toluenediamine is advantageously carried out at a pressure in the range from 1 to 50 bar, preferably in the range from 30 to 45 bar, and at a temperature in the range from 120 to 250° C., preferably at a temperature in the range from 180 to 230° C.

The pressure is preferably selected so that the offgas of the hydrolysis reactor preferably comprises only carbon dioxide, ammonia and inert gases and water and other amines (apart from ammonia) remain in the reactor. The mass ratio of the stream comprising tolylene diisocyanate to water in the reactor is preferably from 4.8:1 to 1:5, preferably from 1:1.0 to 1:3.

The product stream comprising toluenediamine produced in the reaction is preferably separated by distillation into toluenediamine and water in a subsequent step. The distillation can be carried out in any distillation column, with preference being given to using an atmospheric pressure column with internals, e.g. a packed column. The distillation is preferably carried out at a temperature at the bottom in the range from 150 to 200° C., in particular in the range from 170 to 190° C., and a temperature at the top in the range from 95 to 105° C., in particular in the range from 98 to 102° C., at a pressure in the range from 0.9 to 1.1 bar, preferably in the range from 0.95 to 1.05 bar.

The reactor used for the hydrolysis is preferably a residence reactor, for example an at least partially backmixed reactor, preferably a fully backmixed reactor. An example of a suitable reactor is a stirred tank reactor. Further suitable reactors are, for example, tube reactors, jet loop reactors, gas recycle reactors, a reactor having a reaction mixing pump, a reactor having a pump circuit with a static mixer and/or a reactor having a two-fluid mixing nozzle. Preference is given to continuous flow occurring through the reactor used.

To improve the reaction yield, it is also possible to configure the continuously operated, backmixed reactor as a reactor cascade or as a combination of a backmixed prereactor and an unbackmixed after-reactor, for example as a stirred vessel with a downstream tube reactor.

Particular preference is given to gas recycle reactors or stirred vessels with an external pump circuit as reactors.

An exemplary embodiment of the invention is shown in the FIGURE and described in more detail in the following description.

The only FIGURE shows a process flow diagram for the process of the invention for working up an isocyanate-comprising stream.

In a reactor 1, isocyanate originating from an isocyanate-comprising stream is hydrolyzed by means of water to the corresponding diamine. For this purpose, a stream 3 comprising from 10 to 50% by mass of isocyanate, which is obtained, for example, as distillation residue in a work-up of isocyanate by distillation, is fed to the reactor 1 by means of a pump 5. The pump 5 is, for example, a multihead positive displacement pump equipped with solids-tolerant inlet and outlet valves on the product side. A piston membrane pump or a hose piston membrane pump, for example, is suitable as multihead positive displacement pump. Further suitable pumps are, for example, gear pumps, screw pumps, swash plate pumps.

The isocyanate-comprising stream 3 conveyed by the pump 5 goes into a recycle stream 7 which is recirculated to the reactor 1. The temperature necessary for the hydrolysis in the reactor 1 is set by means of a heat exchanger 9 arranged in the recycle stream 7.

The recycle stream 7 is a substream of a crude product stream 11 taken off from the reactor 1. Part of the crude product stream 11, preferably from 90 to 98% by volume, is recirculated as recycle stream 7 to the reactor 1, and the remainder is fed as feed to a distillation column 13. In the distillation column 13, water is removed from the crude product stream 11 by distillation, giving an essentially water-free product stream 15 as bottom product. The essentially water-free product stream 15 is, for example, subsequently worked up further in a further distillation to isolate the diamine. The diamine obtained here can, for example, be recirculated to the preparation of isocyanate by phosgenation.

For the purposes of the present invention, essentially water-free means that the proportion of water is not more than 5% by weight, preferably not more than 2% by weight and in particular not more than 1% by weight.

A stream comprising water and low boilers is taken off at the top of the distillation column 13. The stream 17 comprising water and low boilers is subsequently cooled in a condenser 19, with gaseous constituents being separated off and discharged from the process as offgas 21. Condensable material is partly recirculated to the distillation column 13, if desired taken entirely or in part from the process as wastewater 23 and particularly preferably fed in its entirety to a vessel 25. In the vessel 25, a base or an acid can also be added. Preference is given to introducing a base, for example an alkali metal hydroxide and/or alkaline earth metal hydroxide. The addition of the alkali metal hydroxide and/or alkaline earth metal hydroxide, preferably sodium hydroxide, potassium hydroxide, magnesium hydroxide or calcium hydroxide is denoted by the reference numeral 27. In addition, a water condensate 29 is introduced into the vessel 25. The condensate 29 thus replaces the water which has been chemically consumed in the reactor 1, for example by deionized water.

In the vessel 25, base or acid, condensate 29 and water taken off at the top of the distillation column 13 are mixed and pumped via a feed line 31 into the reactor 1.

A gas stream 33 is taken off at the top of the reactor 1. This gas stream, which comprises carbon dioxide formed in the reaction, water vapor and low boilers, is fed to a condenser 35 in which condensable low boilers are condensed and are recirculated to the reactor 1. Carbon dioxide formed in the reaction and further uncondensable constituents are introduced into the offgas 21 and taken off from the process.

According to the invention, the rate at which the isocyanate-comprising stream 3 is introduced without interruption and steadily into the recycle stream 7 and the recycle stream 7 is introduced into the reactor 1 is selected so that the velocity of the stream fed into the reactor 1 is greater than the velocity of the reaction front formed at the point of introduction. This prevents solids, for example ureas, from forming as intermediate in the region of the line for the recycle stream 7 opening into the reactor 1 and in the line supplying the isocyanate-comprising stream 3, from blocking the lines.

The point of introduction of the isocyanate-comprising stream 3 into the recycle stream 7 is preferably configured in the form of a Venturi nozzle. Good mixing of isocyanate-comprising stream 3 with the recycle stream 7 is obtained in this way. The qualitatively good mixing of the isocyanate-comprising stream into an excess of H-acid compounds such as amines and water leads to formation of many, particularly small solid particles, preferably ureas, having a large surface area, which is particularly advantageous for the outcome of the reaction—high conversions in the hydrolysis reaction at moderate residence times and temperatures. As devices for mixing the isocyanate-comprising stream 3 into the recycle stream 7, it is also possible to use other static mixers, for example annular slit nozzles, or dynamic mixers, for example stirrers or rod mixers.

### LIST OF REFERENCE NUMERALS

1 Reactor

3 Isocyanate-comprising stream

5 Pump

7 Recycle stream

9 Heat exchanger

11 Crude product stream

13 Distillation column

15 Product stream

17 Stream comprising water and low boilers

19 Condenser

21 Offgas

23 Wastewater

25 Vessel

27 Addition of alkali metal hydroxide/alkaline earth metal hydroxide

29 Condensate

31 Feed line

33 Overhead stream

35 Condenser

1. A process for working up a stream (3) comprising from 5 to 90% by weight of isocyanate by hydrolysis of the isocyanate by means of water in a backmixed reactor (1), in which
the isocyanate-comprising stream (3) is introduced to the reactor (1) via a first inlet and the water is introduced via a second inlet,
the isocyanate is reacted with the water in the reactor (1) to form the corresponding diamine,
a diamine-comprising product mixture (11) is taken off continuously from the reactor (1) via a product outlet,
wherein part of the product mixture (11) is recirculated to the reactor (1) and the isocyanate-comprising stream (3) is introduced into the recirculated product stream (7), with the isocyanate-comprising stream (3) being introduced continuously without interruption and the molar ratio of H-acid compounds of the hydrolyzate to isocyanate groups of the stream (3) introduced being kept in the range from 10 to 200.

2. The process according to claim 1, wherein the flow rate of the isocyanate-comprising stream (3) introduced is greater than the speed of the reaction front.

3. The process according to claim 1, wherein the isocyanate-comprising stream (3) is conveyed by means of a multihead positive displacement pump (5) having solids-tolerant inlet and outlet valves.

4. The process according to claim 1, wherein an inert barrier liquid is introduced between the reactants during start-up and shutdown.

5. The process according to claim 4, wherein the pressure of the isocyanate-comprising stream (3) is kept above the pressure in the return stream at the point of introduction of the isocyanate.

6. The process according to claim 1, wherein the isocyanate-comprising stream (3) is a distillation residue from isocyanate production.

7. The process according to claim 1, wherein the diamine-comprising product stream (11) is separated by distillation into a diamine-comprising stream and a water-comprising stream.

8. The process according to claim 1, wherein the point of introduction of the isocyanate-comprising stream (3) into the return stream (7) is configured as a Venturi nozzle.

9. The process according to claim 1, wherein the hydrolysis is carried out at a pressure in the range from 1 to 50 bar, preferably in the range from 30 to 45 bar, and at a temperature in the range from 120 to 250° C., preferably at a temperature in the range from 180 to 230° C.

10. The process according to claim 1, wherein the reactor is a stirred tank reactor, a jet loop reactor, a reactor having a reaction mixing pump, a reactor having a pump circuit with a static mixer and/or a reactor having a two-fluid mixing nozzle.

11. The process according to claim 1, wherein the isocyanate is tolylene diisocyanate.
